(19) United States
(12) Plant Patent Application Publication
(10) Pub. No.: US 2010/0218295 P1
(43) Pub. Date: Aug. 26, 2010

(54) Campanula plant named 'Heavenly Bells'

(50) ;

(76) Inventor: Rodolfo Valdoz Bautista, Half Moon Bay, CA (US)

Correspondence Address: Law offices of James R. Cypher, 409 13th Street, 11th Floor, OAKLAND, CA, 94612, US

(21) Appl. No.: 12/380,004

(22) Filed: Feb. 20, 2009

(51) Int. Cl. A01H 5/00

(52) U.S. Cl. PLT/414

(57) ABSTRACT

A new and distinct cultivar of the Campanulaceae family, named ‘Heavenly Bells’, originated as an interspecies cross by pollinating the seeds of an unnamed variety of Campanula incurva with pollen from the commercial variety Campanula Medium ‘Bells of Holland’. The new variety has large, campanulate flowers. The flower petals at maturity are R.H.S. 91A (violet-blue group) at their base, R.H.S 92B (violet-blue group) at their mid portions, and R.H.S 92 C (violet-blue group) at their tips.

### LATIN NAME OF GENUS AND SPECIES OF THE PLANT CLAIMED

The new plant is Campanula incurva×Campanula Medium.

### VARIETAL DENOMINATION

The new plant's varietal denomination is ‘Heavenly Bells’.

### BACKGROUND OF THE INVENTION

The present invention relates to a new and distinct variety of the Campanulaceae family. The new variety is named ‘Heavenly Bells’. The inventor is Rodolfo Valdoz Bautista, a citizen of the United States of America.

### SUMMARY OF THE INVENTION

The present variety, ‘Heavenly Bells’ was developed at a commercial nursery in Half Moon Bay, Calif., and is an interspecies hybrid obtained by cross pollinating flowers of an unnamed variety of Campanula incurva with pollen from the commercial variety Campanula Medium ‘Bells of Holland’.

‘Heavenly Bells’ is characterized by its flower color and large flowers. The flower petals at maturity are R.H.S. 91A (violet-blue group) at their base, R.H.S 92B (violet-blue group) at their mid portions, and R.H.S 92 C (violet-blue group) at their tips. The new variety's flowers are larger than the flowers of the seed parent, the unnamed Campanula incurva.

The new variety possesses the commercially desirable characteristic of a spreading growth habit with dense foliage.

In addition, the new variety also possesses the additional commercially desirable characteristics of having hardy foliage and roots. The foliage of the new variety stays greener longer than the pollen parent Campanula Medium ‘Bells of Holland’. The new variety also has hardier roots that are more resistant to root fungus diseases than the pollen parent Campanula Medium ‘Bells of Holland’.

The inventor has asexually reproduced the new variety at a commercial nursery in Half Moon Bay, Calif. through three successive generations by cuttings, and has found that the combination of characteristics as herein disclosed remain firmly fixed.

### BRIEF DESCRIPTION OF THE DRAWING

The accompanying drawings serve, by color photographic means, to illustrate the new plant variety. The colors are represented as truly as possible using conventional photographic procedures.

FIG. 1 is a color photograph of a single flower of the new variety.

FIG. 2 is a color photograph of the new variety grown in a 4″ pot illustrating the overall appearance and form of the plant, and the abundance of blooms.

FIG. 3 is a color photograph of the new variety grown in a 4″ pot illustrating the overall appearance and form of the plant, and the abundance of blooms.

FIG. 4 is a color photograph of the new variety grown in a 4″ pot illustrating the overall appearance and form of the plant, and the abundance of blooms.

FIG. 5 is a color photograph of a single flower of the new variety.

FIG. 6 is a color photograph of the base of a plant grown in a 4″ pot of the new variety.

FIG. 7 is a color photograph of a number of flowers of the new variety.

FIG. 8 is a color photograph of a single flower of the new variety.

FIG. 9 is a color photograph of a number of flowers of the new variety.

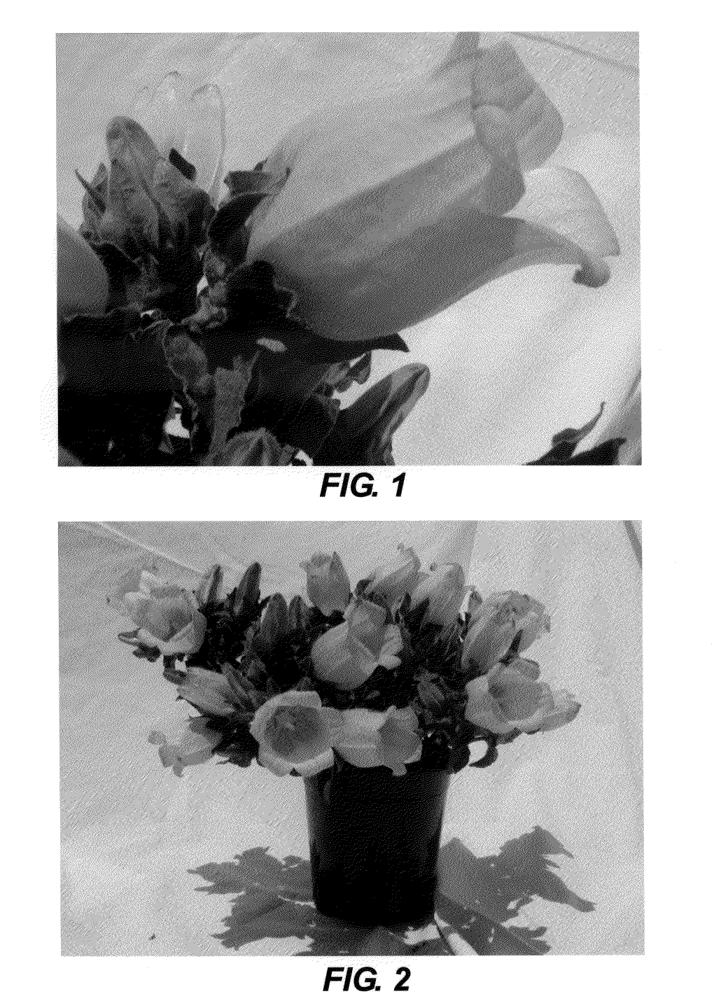

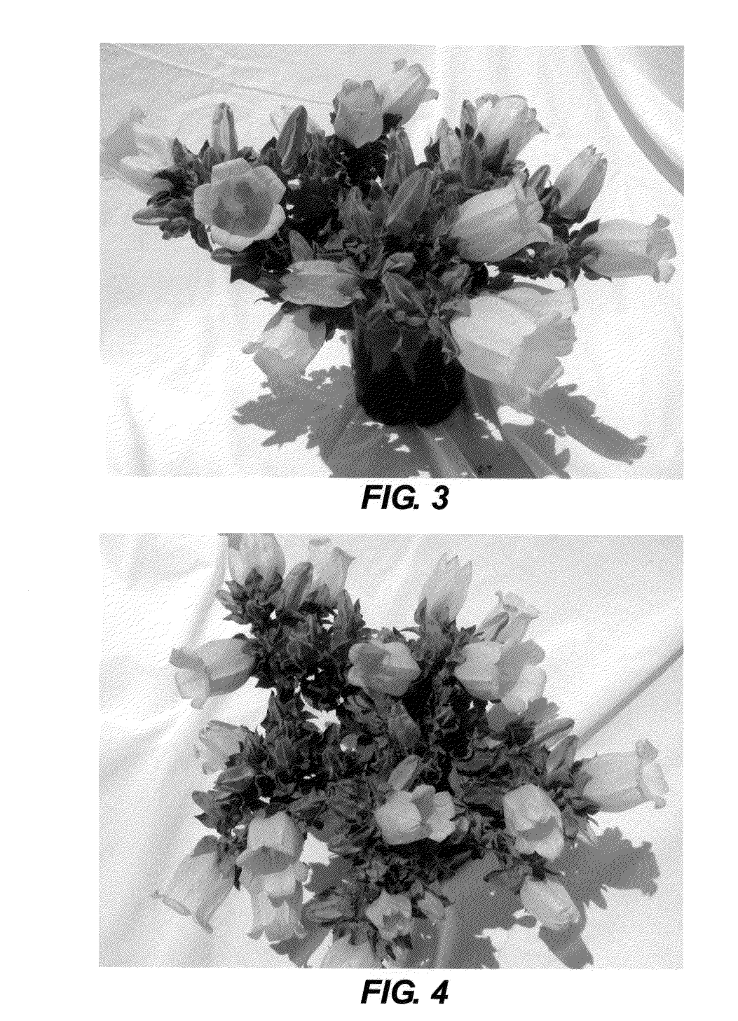

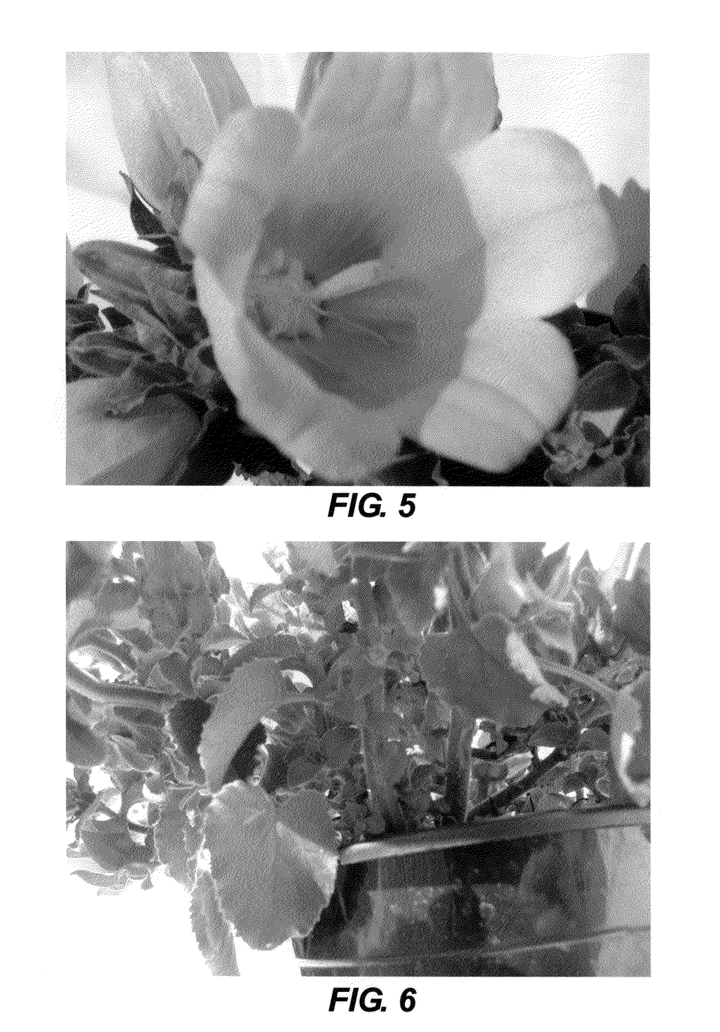

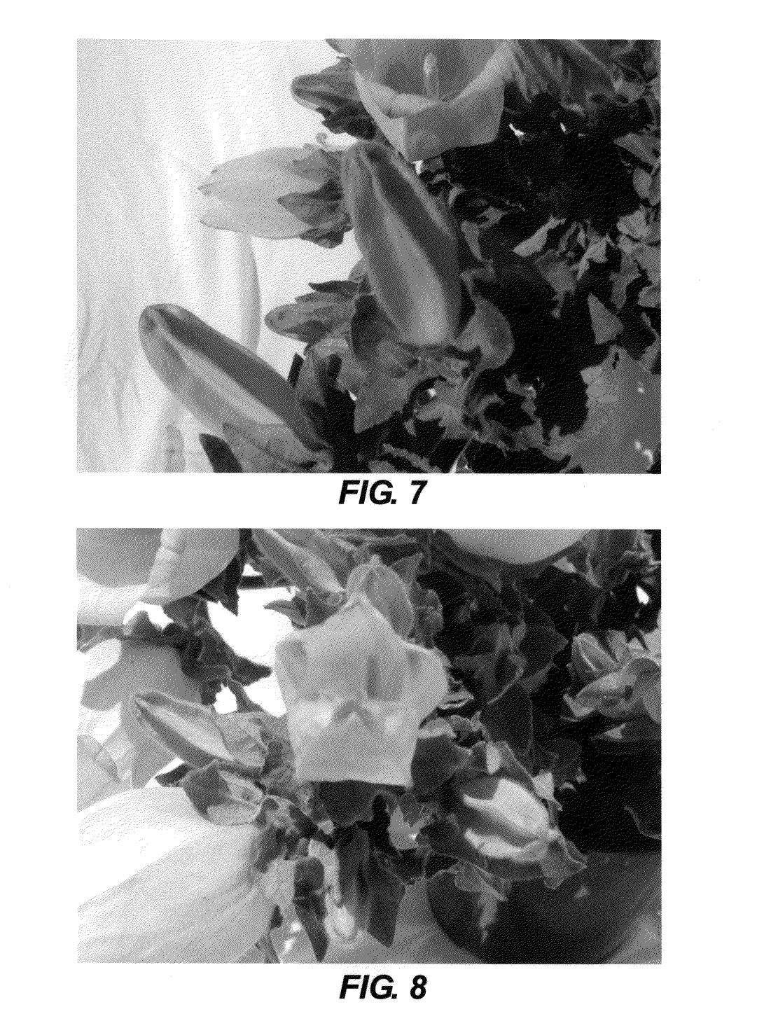

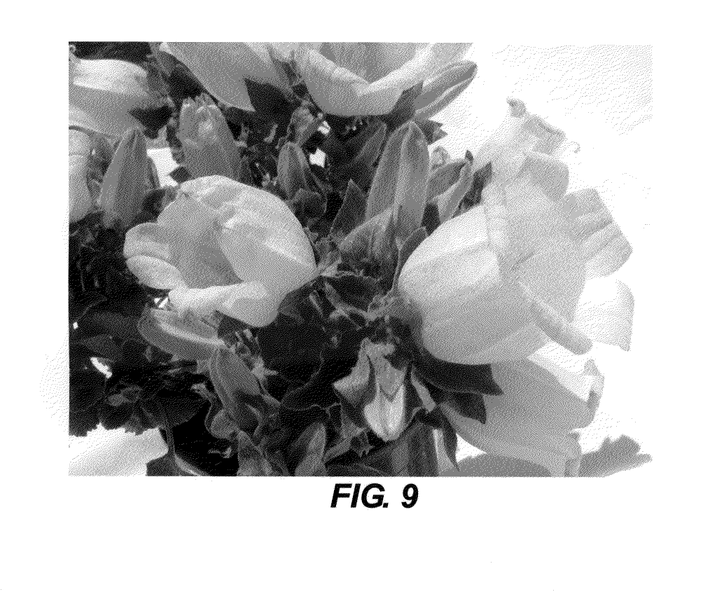

### DETAILED DESCRIPTION OF THE NEW VARIETY

The following is a detailed description of the new variety. The new variety has not been observed under all possible environmental conditions. Color designation and other values stated may deviate slightly from the stated values from flowering to flowering, but the deviations will be within the range expected from varying environmental, seasonal and cultural conditions. Color designations were made according to the R.H.S. Colour Chart published by The Royal Horticultural Society of London, England.

The plant at the time of the observation was grown in a 4 inch pot. The observed plant was approximately 9 months old. The plant had been started indoors in September and was moved out of doors the following April.

The following description is based on observations of optimally fertilized plants grown at a commercial nursery in Half Moon Bay, Calif. Temperatures in Half Moon Bay on average range from 55 to 65 degrees Fahrenheit in the summer months, and from 45 to 55 degrees Fahrenheit in the winter months.

### DETAILED PLANT DESCRIPTION

Varietal name: ‘Heavenly Bells’.
Parentage: Seed parent — Campanula incurva unnamed variety. Pollen parent — Campanula Medium ‘Bells of Holland’.
Classification: Family — Campanulaceae. Genus and Species — Campanula incurva×Campanula Medium.
Form: Terrestrial plant with many spreading lateral stems. Lateral stems are many branched with the plant spreading well away from its pot. Observed plant had a diameter of 27 cm. Height of plants: 9 month old plant is approximately 18 cm high. Stems can range from 15 cm to 20 cm in length.
Stems: General — Stems are many branched with new shoots growing in leaf interfaces. Branches are short — 0.5 cm at first node below terminal flower and 2.5 cm at 7^{th} node below terminal flower. Each lateral branch and each branch from a lateral branch ends in a terminal flower giving the plant a showy appearance. There are as many as three mature flowers on any lateral branch and its sub-branches with a further two immature flowers being close to flowering. Size: Width — 3 mm at base of flowers and as much as 4 mm wide where lateral stems have grown from main stem. Color: Predominately R.H.S. 138 A (yellow-green group). Texture — Puberlent.
Leaves: General — Leaves are whorled on the stem. Very young leaves are entire, and older leaves, farther away from the meristem, becoming very serrate and folded. Mature leaves have acute apexes, sagittate bases and winged petioles, giving the appearance of an arrowhead. Pinnately veined. Size: Length — Mature leaves that are 5 or 6 nodes below the meristem of the lateral branch are approximately 45 mm long including the petiole. The petiole on these leaves is approximately 15 mm long and the blade is 30 to 35 mm long. Leaves closer to the meristem lack petioles or have reduced petioles, and have blades that are 30 to 35 mm long. Width — Mature leaves are approximately 25 to 30 mm wide. Color: Adaxial surface of leaf and petiole is predominately R.H.S. 137 D (yellow-green group), while abaxial surface of leaf and petiole is predominantly R.H.S. 138 B (yellow-green group). Texture — Abaxial surfaces of leaves and petioles are puberlent. Adaxial surfaces of the leaves are puberlent.
Flowers: General — Flowers are actinomorphic, solitary, perfect, terminal and 5-lobed. The calyx is united and lobed. The corolla is campanulate. The stamens have winged bases and uniformly deposit their pollen along much of the length of the style below the stigma.
Sepals: General: The calyx is 5-lobed, regular and united to the ovary with triangular lobes in each sinus between the sepals. Texture is puberulent. Shape: The apex of the sepals are acute and serrate, and the base is lobed between the sinuses. Size: Length — 17 mm from base of petals to tip of sepals when corolla is 40 mm long and flower is mature. When the flower is fully mature the basal appendages are 7 mm long. Width — 16 mm. Color: R.H.S. 137 D (yellow-green group)
Petals: General: Petals inserted on ovary and united to form 5-lobed bell-shaped tube. Texture: Succulent. Glabrous when mature. Petals: There are 5 joined petals. Approximately 15 mm in width. The central ribs of the petals are distinct from the remainder of the petals and short perbulent hairs grow on the ribs. Size of Flower (at full bloom): Length of tube — 60 mm. Diameter of tube — 45 mm. Color: The mature perianth tube (both on its outer and inner surfaces) is R.H.S. 91A at its base and graduating to R.H.S 92B and then to R.H.S 92 C (violet-blue group) near the tips of the petals. Margins and tips of petals are R.HS. 91 A again (violet-blue group). The perianth tube of immature flowers that are just beginning to open is R.H.S. 145 D (yellow-green group).
Androecium (stamens): General: 5 inserted stamens having basal wings that cover and hide the inferior ovary, short filaments and long anthers. The stamens initially stand erect and are stuck to and uniformly spaced around the style by means of their pollen. The stamens become desiccated and highly reflexed above their wings as the flower ages, having deposited their pollen on the style in a uniform blanket just below the stigma before the flower fully matures. Total length of the stamens is 27 mm. Filament: Shape — Winged at base. Basal winged portion is 7 mm tall with short hairs on the wings and this is topped by a thin section to the anther which is 5 to 7 mm tall. Texture — Glabrous. Color — Wings and filament are white R.H.S. 155C (white group). Anther: Shape — Rod shaped. Color — R.H.S. 1444D (yellow-green group). Pollen is R.H.S. 11B (yellow group). Size — 11 mm long.
Gynoecium (pistil): General: Compound, parietal placentation with united style surrounded by an annular diffuse nectary at its insertion. Style: General — Stout and inserted in ovary. Shape — Elongated. Texture — Fleshy and puberlent just below stigma. Size (at full bloom) — Up to 37 mm long. Stigma: General — Inserted at maturity and erect, usually with 3 lobes that become reflexed as flower ages. Size — 6 mm long. Color — R.H.S. 1444D (yellow-green group). Ovary: General — Inferior. Thin epidermis with 3 locules, forming 3 carpels with axile placentation. The ovary is covered by the lobes of the sepals.
Blooming period: The plant normally blooms outside in Half Moon Bay, Calif. in May.
Disease and pest resistance/susceptibility: resists root fungal diseases.
Fragrance: no fragrance.

I claim:

1. A new and distinct variety of Campanula plant, substantially as herein shown and described.
